(19) United States
(12) Patent Application Publication
(10) Pub. No.: US 2025/0101357 A1
(43) Pub. Date: Mar. 27, 2025

(54) INFECTION RECOGNITION AND ELIMINATION IN A MICROFLUIDIC NEURAL LATTICE

(71) Applicant: INTERNATIONAL BUSINESS MACHINES CORPORATION, ARMONK, NY (US)

(72) Inventors: John S. Werner, Fishkill, NY (US); Steven Holmes, Red Hook, NY (US); Benjamin Hardy Wunsch, Mt. Kisco, NY (US); Sagarika Mukesh, Albany, NY (US); Arkadiy O. Tsfasman, Wappingers Falls, NY (US)

(21) Appl. No.: 18/472,703

(22) Filed: Sep. 22, 2023

(51) Int. Cl. C12M 1/12; C12N 5/0793; G06T 7/00; G06V 10/82; G06V 20/69

(52) U.S. Cl. CPC C12M 25/14; C12N 5/0619; G06T 7/0012; G06V 10/82; G06V 20/693; G06T 2207/10056; G06T 2207/20081; G06T 2207/20084; G06T 2207/30024

(57) ABSTRACT

A method, system, and computer program product are provided for monitoring neuron growth within a lattice and stopping a spread of an infection. An infection visual recognition module monitors and captures images of the lattice during the neuron growth. The presence of the infection in the neurons within the lattice is identified. The identifying includes performing, via a deep neural network, visual recognition on the captured images to identify the presence of the infection. In response to the identifying, applying a laser to the infected area of the lattice with sufficient energy for stopping the infection. The lattice is flushed to remove chemical byproducts and dead cells resulting from the laser application.

### BACKGROUND

The present invention relates to computer systems, and more specifically to neural lattices.

Semiconductor memory devices have included synthetic neural networks to mimic biological neural structures. However, synthetic neural networks do not provide the same level of functionality that may be found in biological neural structures. The biological signals obtained by sensors or other devices that are inserted into the biological neural structures typically comprise noisy signals that include information associated with more than one neuron or even millions of neurons.

A microfluidic neural lattice in glass and silicon substrates allows for measurement of the electrical interactions of the precisely arranged neurons. However, neurons growing in this lattice are susceptible to infection.

It would be advantageous to rapidly identify and eliminate infections in a lattice, particularly without operator intervention.

### SUMMARY

A method is provided for monitoring neuron growth within a lattice and stopping a spread of an infection. An infection visual recognition module monitors and captures images of the lattice during the neuron growth. The presence of the infection in the neurons within the lattice is identified. The identifying includes performing, via a deep neural network, visual recognition on the captured images to identify the presence of the infection. In response to the identifying, applying a laser to the infected area of the lattice with sufficient energy for stopping the infection. The lattice is flushed to remove chemical byproducts and dead cells resulting from the laser application.

A computer program product for monitoring neuron growth within a lattice and stopping a spread of an infection, the computer program product. The computer program product comprises a non-transitory tangible storage device having program code embodied therewith, the program code executable by a processor of a computer to perform a method. An infection visual recognition module monitors and captures images of the lattice during the neuron growth. The presence of the infection in the neurons within the lattice is identified. The identifying includes performing, via a deep neural network, visual recognition on the captured images to identify the presence of the infection. In response to the identifying, applying a laser to the infected area of the lattice with sufficient energy for stopping the infection. The lattice is flushed to remove chemical byproducts and dead cells resulting from the laser application.

Embodiments are further directed to a computer system for monitoring neuron growth within a lattice and stopping a spread of an infection comprising one or more processors; a memory coupled to at least one of the processors; a set of computer program instructions stored in the memory and executed by at least one of the processors in order to perform actions of monitoring and capturing images by an infection visual recognition module of a lattice during neuron growth. The presence of the infection in the neurons within the lattice is identified. The identifying includes performing, via a deep neural network, visual recognition on the captured images to identify the presence of the infection. In response to the identifying, applying a laser to the infected area of the lattice with sufficient energy for stopping the infection. The lattice is flushed to remove chemical byproducts and dead cells resulting from the laser application.

The technical effect of embodiments of the present invention is to improve the use of biological neural structures in semiconductor memory devices by rapidly identifying infections in a lattice. This is accomplished by incorporating a microfluidic neural lattice in glass and silicon substrates.

By rapidly identifying infections in the lattice, the infections can be more quickly and efficiently eliminated without user intervention.

### BRIEF DESCRIPTION OF THE SEVERAL VIEWS OF THE DRAWINGS

The subject matter that is regarded as the invention is particularly pointed out and distinctly claimed in the claims at the conclusion of the specification. The foregoing and other features, and advantages of the invention are apparent from the following detailed description taken in conjunction with the accompanying drawings in which:

FIG. 1 illustrates an exemplary network flow diagram, in accordance with one or more aspects of the present invention;

FIG. 2 is a top-down view of the lattice 110 of FIG. 1;

FIG. 2A is a top-down view of an opaque cover plate to control exposure of target wells;

FIG. 3 is a flow chart illustrating monitoring and laser control module;

FIG. 4 illustrates an exemplary deep neural network according to embodiments of the present invention;

FIG. 5 illustrates a backfeeding process that can be used as part of the supervised training; and

FIG. 6 illustrates the operating environment of a computer server embodying a system for infection recognition and elimination in a microfluidic neural lattice.

### DETAILED DESCRIPTION

Synthetic neural networks have been utilized in semiconductor memory devices to mimic biological neural structures. However, synthetic neural networks do not provide the same level of functionality that may be found in biological neural structures. This is because neural structures are often highly complex in nature and are often difficult to characterize effectively.

For example, in a highly complex neural network such as a brain, it is often difficult to isolate, characterize and utilize specific neurons due to the sheer density of biological neural material. Instead, the biological signals obtained by sensors or other devices that are inserted into the biological neural structures typically comprise noisy signals that include information associated with more than one neuron, or even millions of neurons, thereby presenting a challenge to using biological neural structures in a semiconductor memory device.

To combat the challenge of the noisy signals, a microfluidic neural lattice in glass and silicon substrates (referred to as lattice), with chemical, electrical, and magnetic means of stimulation, may allow for measurement of the electrical interactions of the precisely arranged neurons.

Although very useful, neurons growing in this lattice are susceptible to infection. It would be advantageous to rapidly identify and eliminate infections in a lattice, particularly without operator intervention. Embodiments of the present invention provide a system and method for monitoring neuron growth within a lattice, thereby as a result, stopping the spread of the infection. A camera is used to monitor neuron growth within the lattice. Visual recognition is performed on the captured images to identify the presence of an infection. A laser is then applied to the infected and neighboring areas. The lattice is flushed to remove chemical byproducts and dead cells. This allows for rapid identification and elimination of infections in a lattice without operator intervention such that time is not wasted during neuron culture preparation or experimentation.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation, or garbage collection, but this does not render the storage device as transitory because the data is not transitory while it is stored.

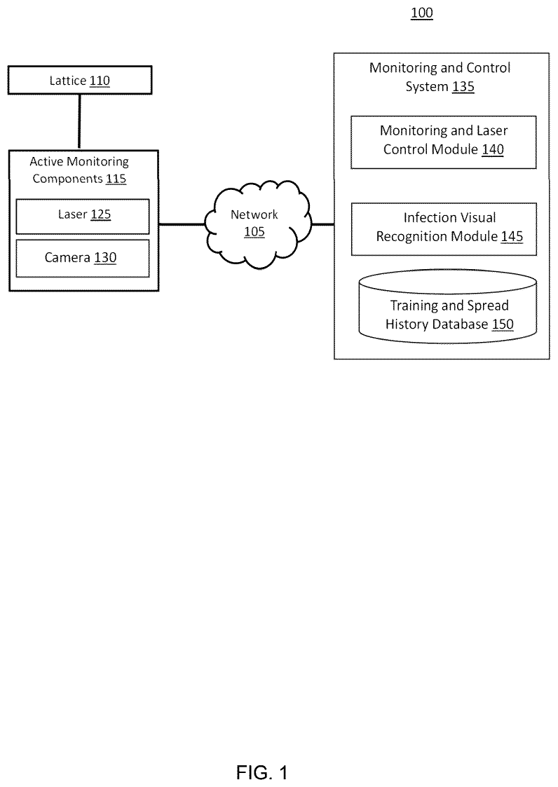

Beginning now with FIG. 1, a network diagram 100 illustrates the components of a system for monitoring neuron growth within a lattice, recognizing the presence of an infection, and stopping the spread of the infection throughout the lattice, according to embodiments of the present invention.

Network diagram 100 is comprised of active monitoring components 115 and monitoring and control system 135. These components are interconnected via wired and/or wireless network 105 and are used to monitor neuron growth and stop the spread of infection within the lattice 110.

The wired and/or wireless network 105 may be any communication protocol that allows data to be transferred between components of the system (e.g., PCIe, I²C, Bluetooth, Wi-Fi, Cellular (e.g., 3G, 4G, 5G), Ethernet, fiber optics, etc.).

The lattice 110 is a patterned, microfluidic template, with chemical, electrical, and magnetic means of stimulation. This provides the lattice 110 with properties, including the ability to load up to 20 μm spherical stem cells into the lattice growth wells and enable growth of axons in an array of connecting microfluidic channels. Additionally, electrical stimulation within the channels is enabled in the lattice 110, as is the ability to measure electrical interactions with measurement electrodes. A continuous flow of nutrients to the neurons, and removal of waste materials are also enabled. As a result, optical monitoring of the neural tissue and an antiseptic environment for neurons are provided.

The active monitoring components 115 are comprised of the laser 125 and the camera 130.

The laser 125 can be any laser that can target the areas of concern where an infection was detected without disrupting the surrounding, uninfected neurons.

In one or more embodiments, the laser 125 is an ultraviolet (UV) laser that is compatible with semiconductor manufacturing. The UV exposure may be in the presence of a chemical that decomposes into toxic material.

In one or more embodiments, the laser 125 is a low temperature laser to help control the thermal budget in the surrounding areas that are not infected.

In one or more embodiments, a targeted cooling unit (not shown) can be used on surrounding areas when the laser 125 is active to maintain a desired temperature for stable neuron growth.

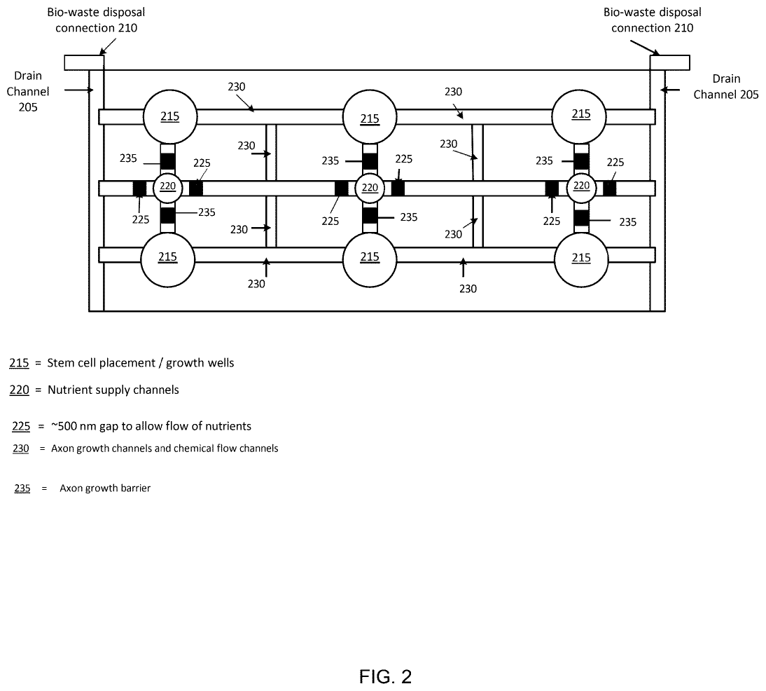

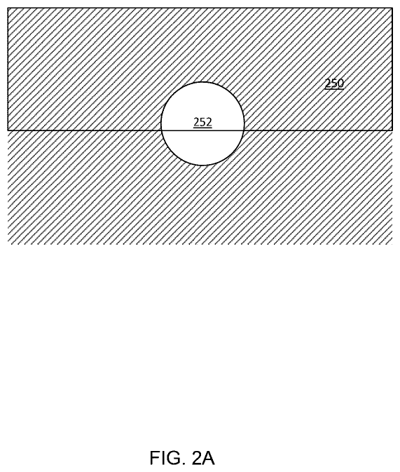

In one or more embodiments an opaque cover plate as shown in FIG. 2A can be automatically controlled to only expose the one or more target wells for the laser such that surrounding areas are protected from accidental exposure. The center 252 is optimally transparent to allow the laser to penetrate to the well, while the remainder of the cover plate 250 is opaque. The size of the center 252 is the size and shape of a well, here ideally circular because the wells are circular. A robotic arm can operate the cover plate 250 to slide it over the top glass cover of the substrate. Thus, only certain wells are selectively exposed while others are protected.

The camera 130 captures images and/or video of the lattice 110 and sends output data to the infection visual recognition module 145 component of the monitoring and control system 135. In preferred embodiments, the camera 130 can be mounted directly to a microscope by fitting the camera 130 with an over-eyepiece camera adapter. At least one camera is positioned vertically above the cultures to examine them for infection. Other cameras can be positioned to capture the same infection from alternate views, for example from the sides. This may provide further information such as the depth of the infection. The images are stored in the training and spread history database (database) 150 for future analysis and training the deep neural network 400.

The monitoring and control system 135 is comprised of monitoring and laser control module (control module) 140, infection visual recognition module 145, and the database 150.

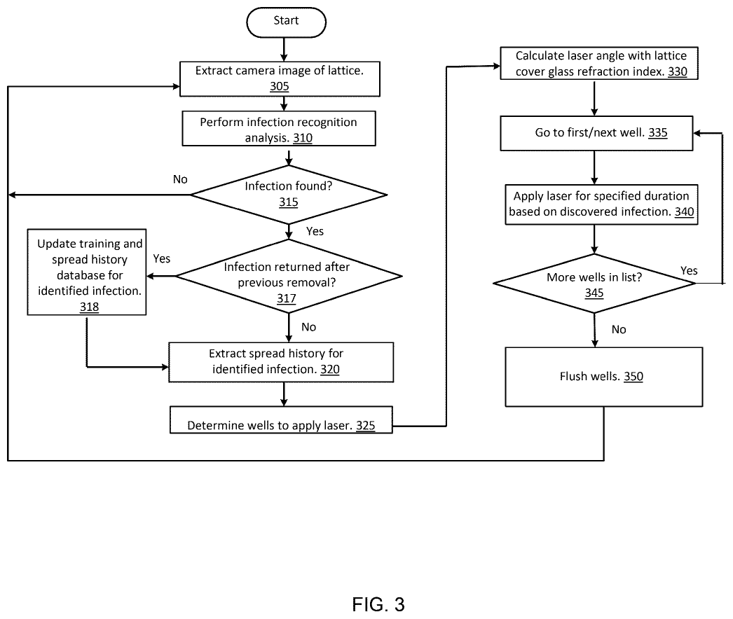

The control module 140 uses the infection visual recognition module 145 and the database 150 to determine when an infection is present, where to direct the beam of the laser 125, and the duration of the laser activation. The monitoring and laser control module 140 also directs the flushing of the lattice The control module 140 is described in further detail with reference to the flow diagram in FIG. 3.

The infection visual recognition module 145, described further in FIG. 3, is an artificial neural network that is trained with supervised learning to visually recognize infections and their spread rate within a culture.

The database 150 stores the training data for the infection visual recognition module 145 which can include images of different types of infections, such as types of molds and information about the speed at which different types of infections spread. The speed of infection refers to how soon the infection spreads from one well to another, which aids the determination of how likely the infection will be removed successfully, or likely cannot be removed at all. The stored images may include those of cultures prior to an infection being recognizable to the human eye. Visual recognition may identify small details that indicate the early signs of an infection.

The speed at which an infection spreads is important data for the control module 140 such that areas around the infection may also be removed with the laser 125 to ensure the infection is eradicated and does not spread to the rest of the culture within lattice 110. If enough training data is available within similar lattices 110, that data should be prioritized in training over the cultures growing in alternative containers. Here, the type of learning model used determines how much data is enough. Ideally, at least three datasets are used: two for training and one for testing. However, even one single dataset can be broken into three parts for the training and the testing.

FIG. 3 shows a flow chart 300 in which the control module 140 uses the infection recognition module 145 and the database 150 to determine when an infection is present, where to direct the laser 125 and the duration for activating the laser. The control module 140 flushes the lattice 110.

At 305 the control module 140 extracts images of the lattice 110 from the camera 130. In preferred embodiments, the camera 130 is located directly above the lattice 110, as in the view presented in FIG. 2. In one or more embodiments, multiple cameras 130 may be used to capture images of the lattice 110 from different angles.

At 310, the infection visual recognition module 145 analyzes the lattice 110.

At 315, the control module 140 uses artificial intelligence image recognition models to analyze the output of the infection visual recognition module 145 to determine if an infection was found. If no infection was found (block 315 “No” branch), the control module 140 returns to block 305 to continue monitoring the lattice 110 as neurons grow. If an infection was found (block 315 “Yes” branch), at 317 the control module 140 determines if the identified infection is one that has returned to the same lattice 110 during the same test sample after a previously attempted removal. An affection may appear as the presence of mold particles in the well where the neurons live. The training data is input to the image processing algorithm to enable the identification. If the identified infection is one that has returned to the same lattice 110 during the same test sample after previously attempted removal (block 317 “Yes” branch), at 318 the control module 140 updates the database 150 for the identified infection. This update to the database 150 indicates that the previous attempt to remove this infection has failed, since the infection returned. This learning step continuously optimizes the model stored in the database 150, because the return of the infection is a sign that the previous removal did not accurately predict the spread of the identified infection within the lattice 110. If the identified infection is not one that has returned to the same lattice during the same test sample after previously attempted removal (block 317 “No” branch), or after executing block 318, the control module 140 continues to block 320 to extract the spread history for identified infection from the database 150. The spread history refers to the spread of the infection throughout the culture, as has been captured during the control module 140 execution. The entry contains details of when a well was infected, when the infection was removed, and whether the infection returned, among other facts.

The database 150 may indicate that characteristics of a given infection may be visually recognizable (e.g., mold particles are visible in the captured image) only in some areas of the lattice 110. Other areas may already be infected, but visual indicators are not yet apparent. Even though not visible, based on training the learning model may predict that a certain number of neighboring wells are likely also infected and must be treated with the laser 125 at least as a precaution. Therefore, the database 150 is used to identify the characteristics such that the infection can be fully irradicated before the entire neuron sample is ruined.

The control module 140 continues to block 325 where it is determined which wells of the lattice 110 needs the application of the laser. The identified wells include the wells where the infection was found and the wells that have a probability above a predetermined threshold (e.g., >50%) where previous spread history indicates the potential that they are also infected.

At 330, the control module 140 calculates the angle at which to direct the laser, taking into account the lattice cover glass refraction index. The refraction index of the glass layer on lattice 110 may impact the angle of the laser prior to reaching the infected neurons.

The lattice glass type and thickness may be uploaded as input to the monitoring and control system 135 prior to use. The inputs include the manufacturer and model. The lattice glass characteristics can also be input by scanning a barcode or QR code to extract lattice information.

In the embodiments described in FIG. 3, there is a single laser whose position can be adjusted to reach any well in the lattice 110. In alternative embodiments, the laser can be in a fixed position and the lattice 110 is moved/shifted to line up the desired wells underneath the laser.

At block 335, the control module 140 begins a loop to iterate over each well 215 where the laser is to be applied.

At block 340, the laser is activated and applied for a specified duration to the current well. In one or more embodiments, the duration may be preprogrammed. In one or more embodiments, the duration may depend on the identified infection.

At block 345, the control module 140 determines if there are more wells in the list to which the laser needs to be applied. If there are more wells in the list (block 345 “Yes” branch), the control module 140 returns to block 335 to go to the next well. If there are no more wells in the list (block 345 “No” branch), the control module 140 proceeds to block 350 to flush the wells to remove chemical byproducts and dead cells by opening a flushing valve (not shown) feeding into the lattice 110. The flushing valve remains open for a duration long enough, e.g., a few seconds, to ensure that all dead neurons that carried the infection are removed from the lattice 110.

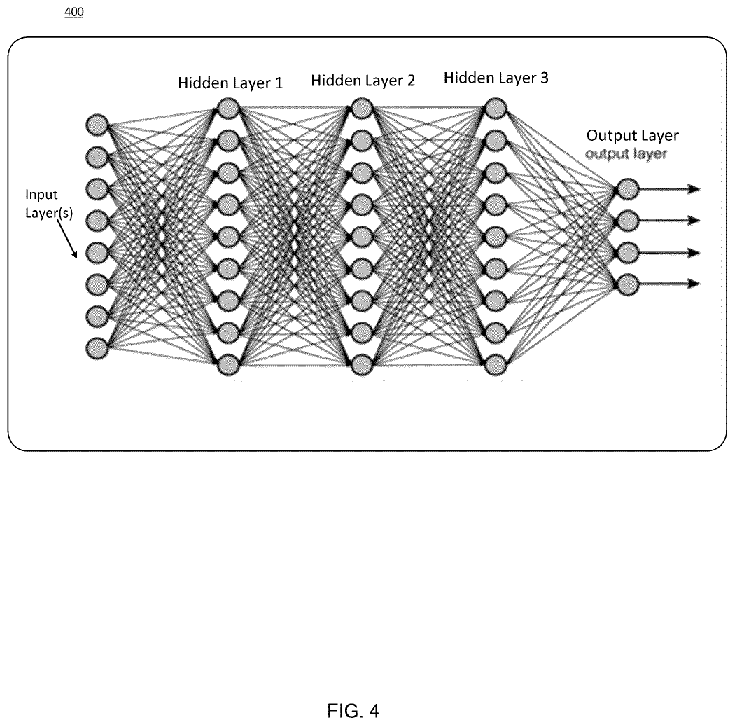

FIG. 4 illustrates an exemplary deep neural network 400 that will be trained using supervised learning to recognize different types of infections and whether they are present in an image of the lattice 110. In practice, the number of inputs, hidden layers, and outputs may differ from the deep neural network as shown in FIG. 4.

In preferred embodiments, the deep neural network is a deep learning convolutional neural network (CNN). The input layer receives images of neuron cultures from the cameras placed on top of the cultures throughout all stages of their lifecycle. In preferred embodiments, the images are images of neuron growth within the lattice 110. In alternate embodiments, the images may be of the same neurons growing in alternate containers, such as a Petri dish.

The neural network of FIG. 4 will have only one input if a single image is being analyzed. Images at different lifecycle stages are analyzed separately (i.e., not all simultaneously input to the neural network as multiple inputs). The neural network of FIG. 4 will have multiple inputs if multiple angles of the same culture are captured simultaneously (e.g., multiple cameras viewing the lattice 110 from different angles).

The convolution operation then extracts different features of the input. The first convolution layer (i.e., hidden layer 1) extracts low-level features such as edges, lines, corners, and colors, whereas higher-level layers (e.g., hidden layers 2, 3, etc.) extract higher-level features (e.g., wells of lattice 110, neurons, axons, infections, etc.).

A CNN works by extracting features from images which eliminates the need for manual feature extraction; the features are not trained, but rather learned while the network trains on a set of images. The CNNs learn feature detection by stacking tens or even hundreds of hidden layers. Pooling layers can be used to reduce the resolution of the features making the features robust against noise and distortion. Pooling will help to account for images of the lattice 110 that are not centered (e.g., lattice is located more towards the top left or the bottom right of the image), if the lattice 110 is tilted in the image. Pooling also accounts for the unpredictable growth direction of neurons within the lattice 110. There may be multiple iterations of convolution followed by pooling within the hidden layers of the CNN.

Nonlinear layers may also be utilized to trigger distinct identification of likely features on each hidden layer. A variety of specific functions such as rectified linear units (ReLUs) and continuous trigger (non-linear) functions may be used to efficiently implement this nonlinear triggering.

Fully connected layers are used as the final layers of the CNN to mathematically sum a weighting of the previous layer of features, indicating the precise mix of “ingredients” to determine a specific target output result (i.e., all the elements of all the features of the previous layer get used in the calculation of each element of each output feature).

Training is performed using a labeled dataset of inputs in a wide assortment of representative input patterns that are tagged with their intended output response. The training data is that which is stored in the database 150. The training dataset is optimally hundreds or even thousands of images cultures throughout at all lifecycle stages.

In one or more embodiments, the labels may include only two outputs (infection and no infections). In one or more embodiments, the labels may include many outputs to indicate the type of infection (e.g., staph infection, mycobacterial infection, fungal infection, etc.)

Labels may additionally include other common features that could be identified within the image (e.g., the lattice 110 wells, the lattice 110 channels, neurons, axons, color, etc.).

Training may also include images prior to a known infection that occurred later which may not be obvious to human eye but can potentially be detected early using visual recognition.

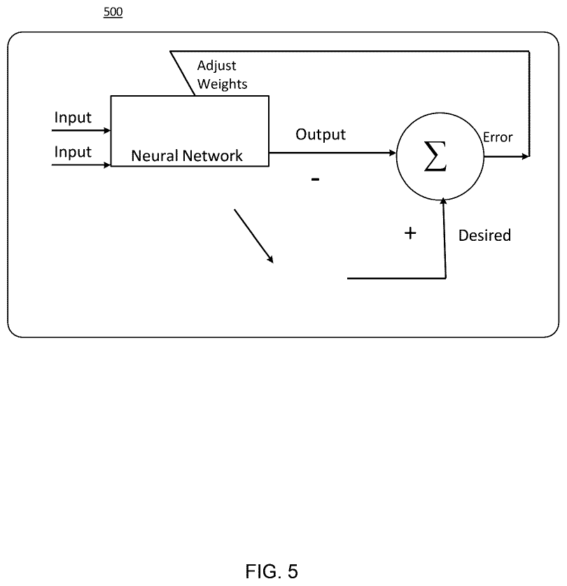

Backfeeding (shown in FIG. 5) can be used as part of the supervised training process where, based on the desired output, the error is determined such that the code libraries can be used to iteratively adjust weights within hidden layers to reduce the error as much as possible (e.g., gradient descent). In one or more embodiments, hyperparameter tuning can be used to manually improve results, for example, by making adjustments to convolution features (number and size), pooling (window size, stride), and fully connected layers (number of neurons within the CNN).

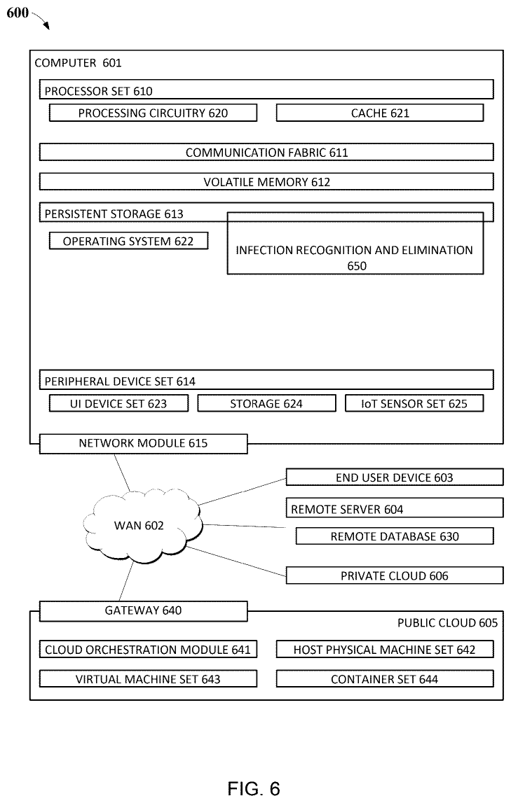

FIG. 6 illustrates an operating environment of a computer server embodying a system for infection recognition and elimination in a microfluidic neural lattice.

Computing environment 600 contains an example of an environment for the execution of at least some of the computer code involved in performing the inventive methods, such as the infection recognition and elimination 650. In addition to block 650, computing environment 600 includes, for example, computer 601, wide area network (WAN) 602, end user device (EUD) 603, remote server 604, public cloud 605, and private cloud 606. In this embodiment, computer 601 includes processor set 610 (including processing circuitry 620 and cache 621), communication fabric 611, volatile memory 612, persistent storage 613 (including operating system 622 and block 650, as identified above), peripheral device set 614 (including user interface (UI), device set 623, storage 624, and Internet of Things (IoT) sensor set 625), and network module 615. Remote server 604 includes remote database 630. Public cloud 605 includes gateway 640, cloud orchestration module 641, host physical machine set 642, virtual machine set 643, and container set 644.

COMPUTER 601 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 630. As is well understood in the art of computer technology, and depending upon the technology, performance of a computer-implemented method may be distributed among multiple computers and/or between multiple locations. On the other hand, in this presentation of computing environment 600, detailed discussion is focused on a single computer, specifically computer 601, to keep the presentation as simple as possible. Computer 601 may be located in a cloud, even though it is not shown in a cloud in FIG. 6. On the other hand, computer 601 is not required to be in a cloud except to any extent as may be affirmatively indicated.

PROCESSOR SET 610 includes one, or more, computer processors of any type now known or to be developed in the future. Processing circuitry 620 may be distributed over multiple packages, for example, multiple, coordinated integrated circuit chips. Processing circuitry 620 may implement multiple processor threads and/or multiple processor cores. Cache 621 is memory that is located in the processor chip package(s) and is typically used for data or code that should be available for rapid access by the threads or cores running on processor set 610. Cache memories are typically organized into multiple levels depending upon relative proximity to the processing circuitry. Alternatively, some, or all, of the cache for the processor set may be located “off chip.” In some computing environments, processor set 610 may be designed for working with qubits and performing quantum computing.

Computer readable program instructions are typically loaded onto computer 601 to cause a series of operational steps to be performed by processor set 610 of computer 601 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 621 and the other storage media discussed below. The program instructions, and associated data, are accessed by processor set 610 to control and direct performance of the inventive methods. In computing environment 600, at least some of the instructions for performing the inventive methods may be stored in block 650 in persistent storage 613.

COMMUNICATION FABRIC 611 is the signal conduction paths that allow the various components of computer 601 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 612 is any type of volatile memory now known or to be developed in the future. Examples include dynamic type random access memory (RAM) or static type RAM. Typically, the volatile memory is characterized by random access, but this is not required unless affirmatively indicated. In computer 601, the volatile memory 612 is located in a single package and is internal to computer 601, but, alternatively or additionally, the volatile memory may be distributed over multiple packages and/or located externally with respect to computer 601.

PERSISTENT STORAGE 613 is any form of non-volatile storage for computers that is now known or to be developed in the future. The non-volatility of this storage means that the stored data is maintained regardless of whether power is being supplied to computer 601 and/or directly to persistent storage 613. Persistent storage 613 may be a read only memory (ROM), but typically at least a portion of the persistent storage allows writing of data, deletion of data and re-writing of data. Some familiar forms of persistent storage include magnetic disks and solid-state storage devices. Operating system 622 may take several forms, such as various known proprietary operating systems or open-source Portable Operating System Interface type operating systems that employ a kernel. The code included in block 600 typically includes at least some of the computer code involved in performing the inventive methods.

PERIPHERAL DEVICE SET 614 includes the set of peripheral devices of computer 601. Data communication connections between the peripheral devices and the other components of computer 601 may be implemented in various ways, such as Bluetooth connections, Near-Field Communication (NFC) connections, connections made by cables (such as universal serial bus (USB) type cables), insertion type connections (for example, secure digital (SD) card), connections made through local area communication networks and even connections made through wide area networks such as the internet. In various embodiments, UI device set 623 may include components such as a display screen, speaker, microphone, wearable devices (such as goggles and smart watches), keyboard, mouse, printer, touchpad, game controllers, and haptic devices. Storage 624 is external storage, such as an external hard drive, or insertable storage, such as an SD card. Storage 624 may be persistent and/or volatile. In some embodiments, storage 624 may take the form of a quantum computing storage device for storing data in the form of qubits. In embodiments where computer 601 is required to have a large amount of storage (for example, where computer 601 locally stores and manages a large database) then this storage may be provided by peripheral storage devices designed for storing very large amounts of data, such as a storage area network (SAN) that is shared by multiple, geographically distributed computers. IoT sensor set 625 is made up of sensors that can be used in Internet of Things applications. For example, one sensor may be a thermometer and another sensor may be a motion detector.

NETWORK MODULE 615 is the collection of computer software, hardware, and firmware that allows computer 601 to communicate with other computers through WAN 602. Network module 615 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 615 are performed on the same physical hardware device. In other embodiments (for example, embodiments that utilize software-defined networking (SDN)), the control functions and the forwarding functions of network module 615 are performed on physically separate devices, such that the control functions manage several different network hardware devices. Computer readable program instructions for performing the inventive methods can typically be downloaded to computer 601 from an external computer or external storage device through a network adapter card or network interface included in network module 615.

WAN 602 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, an administrator that operates computer 601), and may take any of the forms discussed above in connection with computer 601. For example, EUD 603 can be the external application by which an end user connects to the control node through WAN 602. In some embodiments, EUD 603 may be a client device, such as thin client, heavy client, mainframe computer, desktop computer and so on.

REMOTE SERVER 604 is any computer system that serves at least some data and/or functionality to computer 601. Remote server 604 may be controlled and used by the same entity that operates computer 601. Remote server 604 represents the machine(s) that collect and store helpful and useful data for use by other computers, such as computer 601. For example, in a hypothetical case where computer 601 is designed and programmed to provide a recommendation based on historical data, then this historical data may be provided to computer 601 from remote database 630 of remote server 604.

PUBLIC CLOUD 605 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 605 is performed by the computer hardware and/or software of cloud orchestration module 641. The computing resources provided by public cloud 605 are typically implemented by virtual computing environments that run on various computers making up the computers of host physical machine set 642, which is the universe of physical computers in and/or available to public cloud 605. The virtual computing environments (VCEs) typically take the form of virtual machines from virtual machine set 143 and/or containers from container set 644. It is understood that these VCEs may be stored as images and may be transferred among and between the various physical machine hosts, either as images or after instantiation of the VCE. Cloud orchestration module 641 manages the transfer and storage of images, deploys new instantiations of VCEs and manages active instantiations of VCE deployments. Gateway 640 is the collection of computer software, hardware, and firmware that allows public cloud 605 to communicate through WAN 602.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 606 is similar to public cloud 605, except that the computing resources are only available for use by a single enterprise. While private cloud 606 is depicted as being in communication with WAN 602, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 605 and private cloud 606 are both part of a larger hybrid cloud.

What is claimed is:

1. A method for monitoring neuron growth within a lattice and stopping a spread of an infection, the method comprising:
monitoring and capturing images by an infection visual recognition module of the lattice during the neuron growth;
identifying a presence of the infection in the neurons within the lattice, wherein the identifying comprises:
performing, via a deep neural network, visual recognition on the captured images to identify the presence of the infection;
in response to the identifying, applying a laser to an infected area of the lattice with sufficient energy for stopping the infection; and
flushing the lattice to remove chemical byproducts and dead cells resulting from the infected area following the applying the laser.

2. The method of claim 1, wherein a camera mounted on a microscope monitors the neuron growth.

3. The method of claim 1, wherein an angle for applying the laser depends on a refraction index of a lattice cover glass and a refraction index of a lattice glass layer.

4. The method of claim 1, wherein an infection spread history is tracked by the infection visual recognition module using the captured images.

5. The method of claim 4, wherein the infection visual recognition module includes the infection spread history to determine where to apply the laser such that neighboring wells to a plurality of identified infection wells are targeted even if there are no visual signs of infection.

6. The method of claim 1, wherein the deep neural network is a convolutional neural network, and wherein the deep neural network performs the visual recognition.

7. The method of claim 1, wherein the deep neural network is trained to recognize one or more of a plurality of types of infections, lattice wells, lattice channels, color, neurons, and axons.

8. The method of claim 1, wherein a supervised training includes backfeeding to iteratively adjust weights within hidden layers of the deep neural network.

9. A computer program product for monitoring neuron growth within a lattice and stopping a spread of an infection, the computer program product comprising a non-transitory tangible storage device having program code embodied therewith, the program code executable by a processor of a computer to perform a method, the method comprising:
monitoring and capturing images by an infection visual recognition module of the lattice during the neuron growth;
identifying a presence of the infection in the neurons within the lattice, wherein the identifying comprises:
performing, via a deep neural network, visual recognition on the captured images to identify the presence of the infection;
in response to the identifying, applying a laser to an infected area of the lattice with sufficient energy for stopping the infection; and
flushing the lattice to remove chemical byproducts and dead cells resulting from the infected area following the applying the laser.

10. The computer program product of claim 9, wherein a camera mounted on a microscope monitors the neuron growth.

11. The computer program product of claim 9, wherein an angle for applying the laser depends on a refraction index of a lattice cover glass and a refraction index of a lattice glass layer.

12. The computer program product of claim 9, wherein an infection spread history is tracked by the infection visual recognition module using the captured images.

13. The computer program product of claim 12, wherein the infection visual recognition module includes the infection spread history to determine where to apply the laser such that neighboring wells to a plurality of identified infection wells are targeted even if there are no visual signs of infection.

14. The computer program product of claim 9, wherein the deep neural network is a convolutional neural network, and wherein the deep neural network performs the visual recognition.

15. The computer program product of claim 9, wherein the deep neural network is trained to recognize one or more of a plurality of types of infections, lattice wells, lattice channels, color, neurons, and axons.

16. The computer program product of claim 9, wherein a supervised training includes backfeeding to iteratively adjust weights within hidden layers of the deep neural network.

17. A computer system for monitoring neuron growth within a lattice and stopping a spread of an infection, the computer system comprising:
one or more processors;
a memory coupled to at least one of the processors;
a set of computer program instructions stored in the memory and executed by at least one of the processors in order to perform actions of:
monitoring and capturing images by an infection visual recognition module of the lattice during the neuron growth;
identifying a presence of the infection in the neurons within the lattice, wherein the identifying comprises:
performing, via a deep neural network, visual recognition on the captured images to identify the presence of the infection;
in response to the identifying, applying a laser to an infected area of the lattice with sufficient energy for stopping the infection; and
flushing the lattice to remove chemical byproducts and dead cells resulting from the infected area following the applying the laser.

18. The computer system of claim 17, wherein a camera mounted on a microscope monitors the neuron growth.

19. The computer system of claim 17, wherein an angle for applying the laser depends on a refraction index of a lattice cover glass and a refraction index of a lattice glass layer.

20. The computer system of claim 17, wherein an infection spread history is tracked by the infection visual recognition module using the captured images; and
wherein the infection visual recognition module includes the infection spread history to determine where to apply the laser such that neighboring wells to a plurality of identified infection wells are targeted even if there are no visual signs of infection.
